(19) United States
(12) Patent Application Publication
(10) Pub. No.: US 2004/0019379 A1
(43) Pub. Date: Jan. 29, 2004

(54) Intracorneal lens with flow enhancement area for increased nutrient transport

(75) Inventors: Robert Glick, Lake Forest, CA (US); Daniel G. Brady, San Juan Capistrano, CA (US)

Correspondence Address: Frank J. Uxa, Suite 300, 4 Venture, Irvine, CA, 92618, US

(73) Assignee: Advanced Medical Optics, Inc.

(21) Appl. No.: 10/205,705

(22) Filed: Jul. 25, 2002

(51) Int. Cl. A61F 2/14

(52) U.S. Cl. 623/5.13; 623/5.16

(57) ABSTRACT

Intracomeal lenses having flow enhancement regions facilitate optimized nutrient transmission from posterior to anterior sides of lenses. Thinning, fenestration, and related structural emplacements permit, for example, hyperopic lenses to be crafted whereby nutrient transport is substantially enhanced in novel ways.

### BACKGROUND OF THE INVENTION

The present invention relates to intracorneal lenses. More particularly, the invention relates to an intracorneal lens formed with a flow enhancement portion for improving nutrient transport through the thickest portion of the lens.

Various treatments are known for correcting corneal refractive errors. The use of lasers, for instance, to reshape the cornea by removing corneal tissue, has become increasingly popular in recent years. However, the removal of tissue can result in loss of the structural integrity of the cornea, and can also cause bulging. Furthermore, once corneal tissue has been removed, it can not easily be restored. Thus, laser vision correction is substantially irreversible.

The need for a reversible treatment which does not adversely affect the structural integrity of the cornea has led to the use of intra-corneal implants, which do not require the removal of tissue. Instead, a single small incision is made in the cornea to make a flap or hinge, which is then folded back to expose the middle layer of corneal tissue known as the stromal bed. A corrective lens, typically formed of hydrogel material, is placed on the stromal layer. Then the flap is returned to its initial position and smoothed over the lens.

Ideally, an intraocular lens should be made from a material having a relatively high index of refraction relative to the corneal stroma (i.e. greater than 1.45) and high permeability to water soluble nutrients, such as glucose, that are critical for maintaining optical health. Unfortunately, an ideal material having both these characteristics has yet to be found. Many high refractive index materials, such as polysulfone and PMMA, have been found to be insufficiently permeable, and could possibly cause nutritional stress leading to nebular opacification, anterior corneal necrosis, and other complications. On the other hand, many materials having higher permeability have lower refractive indices and less satisfactory optical qualities. Still other highly permeable materials require complex and expensive manufacturing processes.

Attempts have been made in the past to improve nutrient transfer through corneal onlays or implants by providing a lens with one or more openings allowing nutrients to pass from the posterior side of the lens to the anterior side. U.S. Pat. No. 4,624,669 to Grendahl, for instance, discloses an intracorneal lens formed of polysulfone or PMMA, and having a plurality of pin holes or pores either positioned about the edge of the lens or randomly spaced about the entire surface area of the lens. U.S. Pat. No. 4,646,720 to Peyman et al. discloses an intracorneal lens having either a single, relatively large (up to about 64% of the total optical area of the lens) opening formed at the center of the lens or a plurality of randomly distributed smaller openings. U.S. Pat. No. 6,102,946 to Nigam discloses intracorneal lenses formed from microporous hydrogel material.

Unfortunately, none of the prior art attempts discussed above have been entirely successful in providing an economically manufactured intracorneal lens in which both optical qualities and nutrient transfer are optimized.

Therefore, it would be advantageous to develop an intraocular lens having a flow enhancement region which allows nutrients to pass from the posterior side of the lens to the anterior side without interfering with the optical qualities of the lens and without requiring complex or costly manufacturing processes.

### SUMMARY OF THE INVENTION

In accordance with the present invention, new intracorneal lenses have been designed with a flow enhancement region for allowing more effective transmission of nutrients from a posterior to an anterior side of a lens.

In one broad aspect of the invention, the flow enhancement region comprises a thinned region in the thickest portion of the lens. The thinned region comprises a small surface area relative to the total optical area of the lens. In an especially preferred embodiment of the invention, the thinned region comprises from about 1% to about 5% of the total optical area of the lens. Preferably the thinned region comprises a gradual reduction in thickness to minimize such problems as glare, light scattering and reduction in optical image quality.

In another broad aspect of the invention, the flow enhancement region comprises a fenestrated region in the thickest portion of the lens. The fenestrated region may consist of a single opening or a plurality of openings, and preferably comprises from about 1% to about 5% of the total optical area of the lens. Still more preferably, the walls of the opening or openings are angled to control the direction in which light is reflected.

Each and every feature described herein, and each and every combination of two or more of such features, is included within the scope of the present invention provided that the features included in such a combination are not mutually inconsistent.

Additional aspects, features, and advantages of the present invention are set forth in the following description and claims, particularly when considered in conjunction with the accompanying drawings in which like parts bear like reference numbers.

### BRIEF DESCRIPTION OF THE DRAWINGS

FIG. 1 is a plan view of an intracorneal lens according to a preferred embodiment of the invention;

FIG. 2 is a sectional view taken through line 2-2 of FIG. 1;

FIG. 3 is a plan view of an intracorneal lens according to an alternate embodiment of the invention;

FIG. 4 is a sectional view taken through line 4-4 of FIG. 3;

FIG. 5 is a plan view of an intracorneal lens according to yet another alternate embodiment of the invention; and

FIG. 6 is a sectional view taken through line 6-6 of FIG. 5.

### DETAILED DESCRIPTION OF THE PREFERRED EMBODIMENTS

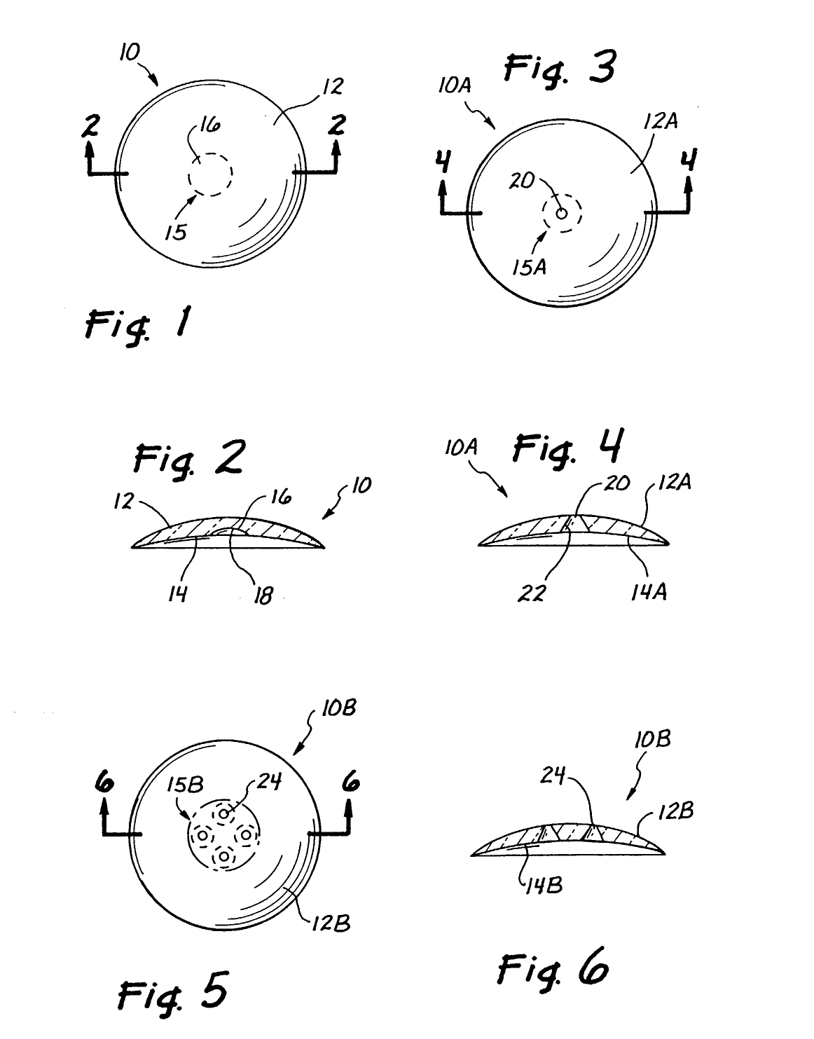

Referring now to the drawings, FIGS. 1-6 show various embodiments of an intracorneal lens 10 according to the present invention. Each of the illustrated intracorneal lenses is a hyperopic lens designed for the correction of far-sighted vision. As such, each intracorneal lens 10 includes an anterior surface 12 that is convex approaching the optical axis and a posterior surface 14 that is concave approaching the optical axis. In addition, each intracorneal lens 10 is thickest at its center and thinnest at its peripheral edge.

In conventional hyperopic lenses, the increased thickness of the center results in reduced nutrient flow through the center of the lens, and a relatively large edge-to-center nutrient gradient. Edge-to-center nutrient gradients are not typically a concern in intracorneal lenses for the treatment of other types of vision problems such as myopia or astigmatism. Nonetheless, the teachings disclosed herein could easily be adapted to such lenses if needed. Accordingly, although these teachings are particularly beneficial in connection with hyperopic lenses, other types of lenses are included within the scope of the invention.

Referring more specifically to FIGS. 1 and 2, the intracorneal lens 10, comprises a lens body which may be formed of any optical material, preferably a hydrogel material, that is permeable or semi-permeable to water soluble nutrients such as glucose. The lens body includes a thinned central region 16 having a sufficiently small surface area relative to the total optical surface area to minimize light scattering. Both the thickness and the diameter of the thinned region depend on a variety of factors including the lens diameter, diopter power and water content of the lens material. Preferably, however, this diameter is selected such that the surface area of the thinned region 16 comprises about 1% to about 5% of the total optic area of the intracorneal lens 10. For instance, in an intracorneal lens having a diameter of 5.0 mm, the thinned area 16 could be limited to the central 0.5 mm, which represents exactly 1% of the projected surface area of the intracorneal lens 10.

The thinned region 16 preferably comprises a gradual reduction in thickness approaching the center of the lens. This gradual reduction results in a reduction of light scattering and other visual symptoms relative to an abrupt reduction. In the illustrated embodiment, the thinned region 16 is created by forming an arcuate indentation 18 in the posterior surface 14 of the intracorneal lens.

In a second embodiment of the invention, illustrated in FIGS. 3 and 4, a single opening or fenestration 20 is formed through the center of the intraocular lens 10A. The diameter of the opening 20, like the diameter of the thinned area in the previous embodiment, depends on factors such as the lens diameter, diopter power, and water content of the lens material but should comprise from about 1% to about 5% of the total optical area of the intraocular lens 10A.

The opening 20 preferably includes an angled sidewall 22 that slopes radially inwardly toward the center of the intraocular lens 10A. The angle of the sidewall 22 can be selected to control the direction in which light is reflected, and thus to minimize glare, scattering and other undesirable optical effects.

In the embodiment of FIGS. 5 and 6, the single opening is replaced by a plurality of smaller openings 24 clustered together in a fenestrated zone or region 26 at or near the thickest section, i.e. the center, of the intraocular lens 10B. Once again, the total surface area of the fenestrated region preferably comprises from about 1% to about 5% of the total optical surface area. Furthermore, while the illustrated embodiment shows four circular openings that are generally equally spaced from the center, and provided at generally equal radial intervals from one another, the number, shape, and arrangement of the openings may be altered without departing from the principles of the invention.

While this invention has been described with respect to various specific examples and embodiments, it is to be understood that the invention is not limited thereto and that it can be variously practiced within the scope of the following claims.

1. A lens configured for implantation into the cornea of a patient, the lens comprising:
an optical body having a posterior side, an anterior side, and a portion between the posterior and anterior sides defining a thickness, the thickness varying from a minimum at a first portion of the body to a maximum at a second portion of the body, the optical body being formed of nutrient-permeable material for allowing a flux of nutrients from the posterior side to the anterior side across the optical body and defining a nutrient gradient between the first and second portions of the body; and
means formed in the optical body for reducing the nutrient gradient.

2. The lens according to claim 1, wherein:
the lens includes a center and an edge;
the lens is configured for correcting the vision of a hyperopic patient;
the first portion of the body comprises the edge of the lens; and
the second portion of the body comprises the center of the lens.

3. The lens according to claim 1, wherein the means for reducing the nutrient gradient comprises at least one thinned area formed closer to the second portion of the body than to the first portion of the body.

4. The lens according to claim 3, wherein the at least one thinned area is formed in the second portion of the body.

5. The lens according to claim 3, wherein the optical body comprises a total optical area, and wherein the at least one thinned area comprises a sufficiently small portion of the total optical area to minimize interference with the patient's vision.

6. The lens according to claim 5, wherein the at least one thinned area comprises from about 1% to about 5% of the total optical area.

7. The lens according to claim 3, wherein the at least one thinned area comprises an edge portion and a center portion, with a gradual reduction in thickness from the edge portion to the center portion.

8. The lens according to claim 7, wherein the at least one thinned area comprises at least one arcuate indentation in the posterior side of the lens body.

9. The lens according to claim 1, wherein the means for reducing the nutrient gradient comprises a fenestrated area formed closer to the second portion of the body than to the first portion of the body.

10. The lens according to claim 9, wherein the fenestrated area comprises a single opening extending through the lens body.

11. The lens according to claim 9, wherein the fenestrated area comprises a plurality of openings extending through the lens body.

12. The lens according to claim 10, wherein the opening includes an angled sidewall for reducing optical aberrations and improving optical quality of the lens.

13. The lens according to claim 11, wherein at least one of the openings includes an angled peripheral wall for reducing optical aberrations and improving optical quality of the lens.

14. The lens according to claim 9, wherein the fenestrated area is formed in the second portion of the body.

15. The lens according to claim 9, wherein the optical body comprises a total optical area, and wherein the fenestrated area comprises a sufficiently small portion of the total optical area to minimize interference with the patient's vision.

16. The lens according to claim 15, wherein the fenestrated area comprises from about 1% to about 5% of the total optical area.

17. The lens according to claim 1, wherein the optical body is formed of hydrogel material.

18. The lens according to claim 3, wherein the lens is a monofocal lens.

19. A lens configured for implantation into the cornea of a patient, the lens comprising:
an optical body designed for hyperopic vision correction and being formed of a nutrient-permeable material for allowing a flux of nutrients across the optical body, the flux generally decreasing from the edge of the body to the center of the body to define an edge-tocenter nutrient gradient; and
a flow enhancement area formed in the center of the optical body and configured to increase the flow of nutrients through the center of the lens and decrease the edge-to-center nutrient gradient;
wherein the optical body comprises a total optical area, and the flow enhancement area comprises about 1% to about 5% of the total optical area.
